(19) United States
(12) Patent Application Publication
(10) Pub. No.: US 2019/0002832 A1
(43) Pub. Date: Jan. 3, 2019

(54) HUMANIZED MOUSE MODEL OF MYASTHENIA GRAVIS AND MSC THERAPY

(71) Applicants: ASSOCIATION INSTITUT DE MYOLOGIE, PARIS (FR); INSTITUT NATIONAL DE LA SANTE ET DE LA RECHERCHE MEDICALE, PARIS (FR); SORBONNE UNIVERSITE, PARIS (FR); CENTRE NATIONAL DE LA RECHERCHE SCIENTIFIQUE, PARIS (FR)

(72) Inventors: SONIA BERRIH-AKNIN, CESAREA (IL); MURIEL SUDRES, IVY SUR SEINE (FR)

(73) Assignees: ASSOCIATION INSTITUT DE MYOLOGIE, PARIS (FR); INSTITUT NATIONAL DE LA SANTE ET DE LA REACHERCHE MEDICALE, PARIS (FR); SORBONNE UNIVERSITE, PARIS (FR); CENTRE NATIONAL DE LA RECHERCHE SCIENTIFIQUE, PARIS (FR)

(21) Appl. No.: 16/065,155

(22) PCT Filed: Dec. 23, 2016
(86) PCT No.: PCT/EP2016/082607

§ 371 Date: Jun. 22, 2018

(30) Foreign Application Priority Data
Dec. 24, 2015 (EP) 15307148.5

(51) Int. Cl. C12N 5/0775; A61K 35/28; A01K 67/027

(52) U.S. Cl. CPC C12N 5/0662; A61K 35/28; A01K 2267/0325; C12N 2502/11; A01K 2207/12; A01K 67/0271

(57) ABSTRACT

The present invention relates to an animal model of myasthenia gravis, and to uses thereof.

The present invention relates to an animal model of myasthenia gravis, and to uses thereof.

### BACKGROUND OF THE INVENTION

Acquired Myasthenia Gravis (MG) is a rare autoimmune neuromuscular disease mediated by antibodies (Abs) directed against proteins of the neuromuscular junction (NMJ) leading to a fluctuating skeletal muscle weakness and fatigability. In 85% of patients, autoAbs are specific of the nicotinic acetylcholine receptor (AChR) that trigger the activation of complement system, accumulation of membrane attack complexes, destruction of the post synaptic muscle membrane, reduction in the number of functional AChR and disruption of neuromuscular transmission. The thymus, site of T cell maturation and establishment of central tolerance, is clearly involved in the pathogenesis of the disease. In AChR-seropositive MG patients, the thymus often displays structural and functional abnormalities as thymoma (15%) or thymic follicular hyperplasia (60%) characterized by the presence of ectopic germinal centers (GC). Hyperplastic thymus contains all the components of the anti-AChR immune response: antigen presenting cells (APC) and the autoantigen itself, autoreactive T cells and autoAbs producing-B cells. MG thymus proinflammatory environment is suspected to induce immune dysregulation promoting autoimmune reaction (Berrih-Aknin and Le Panse 2014). Besides, thymectomy, mainly performed in early onset MG patients (EOMG), represents one of the four therapeutic option with (i) acetylcholinesterase inhibitors (symptomatic therapy by improving neuromuscular transmission), (ii) steroids and immunosuppressive agents (generally used for long-term therapy) and (iii) plasmapheresis and intravenous immunoglobulins (to treat acute MG exacerbation). Despite those therapeutic options, MG remains debilitating and problematic to stabilize. Furthermore, steroids and immunosuppressive drugs can cause severe side effects as they are long-term treatments. Thus, more efficient and less toxic treatments need to be developed.

To this purpose relevant animal models that faithfully mimics the human disease are crucial. Current experimental MG models are induced in rodents (i) by AChR immunization (EAMG) but this model presents an inflammatory bias and does not reproduce thymus abnormalities or (ii) by grafting MG thymus tissue under the kidney capsule of immunodeficient SCID mice, but without reproducing clinical weakness and for which human cells could not be detected (Schonbeck, Padberg et al. 1992).

Therefore, a need still exists of a reliable animal model that replicates all features of the human MG disease.

### SUMMARY OF THE INVENTION

In a first aspect, the present invention relates to a method for generating conditioned mesenchymal stem cells, useful for the treatment of an autoimmune disease such as MG, comprising coculturing resting MSCs (or rMSCs) with peripheral blood mononuclear cells (PBMCs) or with monocytes.

In another aspect, the invention relates to a conditioned mesenchymal stem cell (or cMSC) for use in a method for the treatment of an autoimmune disease, in particular for the treatment of MG.

A further aspect of the invention relates to a humanized animal model of Myasthenia Gravis (MG), wherein a human thymic tissue fragment is transplanted subcutaneously in an immunodeficient non-human animal. This humanized animal model advantageously mimics the features of the human disease, thereby allowing a variety of uses such as for identifying new treatments of MG and/or studying functional features of MG.

### LEGENDS TO THE FIGURES

FIG. 1. Characterization of the new MG preclinical model. A. Human AChR-specific Abs were detected in mouse serum. Anti-AChR Abs titers were measured by RIA in the serum of the mice grafted with thymus fragments from CTRL (open circles), seronegative (closed triangles) or seropositive MG patients displaying low (closed squares) or high AChR Abs titers (closed diamonds). Each symbol represents the mean value of Ab titers (nmol/L)±SEM of the different mice (n=2 to 5) included in each experiment. B. Mice displayed MG-like clinical symptoms. The Kaplan Meyer curve shows the occurrence of the disease (score>1) in the high titers seropositive MG group (closed diamonds, n=31), in the low titers seropositive MG group (closed squares, n=18), in the seronegative MG group (closed triangles, n=14), and in the CTRL group (open circles, n=51). C. Endplate AChR content was diminished in MG groups. AChR content of the diaphragmatic muscle were measured by ¹²⁵I-αBGT labeling. Cpm data are normalized using the cpm mean values of the CTRL group (levels set at 100%, white histogram). Histograms correspond to the mean values±SEM in each group (CTRL, n=36; MG low, n=13; MG high, n=22). D. Endplate AChR loss correlated with MG severity. Each symbol represents one mouse from seronegative and both seropositive MG groups. E. Patients Abs titers correlated with mouse Abs titers. Each symbol represents the AChR-specific Abs titer measured in MG patient and the corresponding mean value of Abs titers measured in mice for each experiment. F. Patients score correlated with mouse score. Each symbol represents the score of MG patient and the corresponding mean score attributed in mice for each experiment. G and H. In human and mouse, Abs titers did not correlate with clinical scores.

A and C, p-values were determined according to Student t test. B, p-values were determined according to Log-rank (Mantel-Cox) test. D to H, p-values were determined according to linear regression test.

FIG. 2. Xenogenic thymus fate. A. Picture of the human thymus fragments 2 months after the graft in the mouse's back. B. Hematoxylin/eosin coloration of thymic section. C. keratin and fibronectin labeling of thymic section. D. CD4+ together with CD8+ cells labeling. E. CD21 labeling of thymic section showing GC. F. CD4, CD8, CD20, BAFF, BLIMP1 mRNA expression were analyzed in the xenogenic thymus by q-PCR. G. IL-2, IL-6, IL-17, TNF-α and IFN-γ mRNA expression in the xenogenic thymus.

F and G. CTRL, n=11 (4 experiments); MG, n=19 (6 experiments) and p-values were determined according to t-test.

FIG. 3. Human cells home to the mouse lymphoid organs. A to C. FACS analysis of the expression of CD45 positive cells in the spleen (A, CTRL, n=25: MG, n=26), in the blood (B, CTRL, n=5: MG, n=11) and in the bone marrow (C, CTRL, n=7: MG, n=5) of grafted animals. Six to seven experiments are included and *p-values<0.05 and **p-values<0.01 were determined according to Student t test. A2 and A3. IHC were performed on spleen section showing human cells (laminA/C positive cells, in green).

FIG. 4. Human lymphocytes in spleen of NSG mice. A to D. FACS analysis of the expression of CD4SP (A, CTRL, n=18: MG, n=23), of CD8SP (B, CTRL, n=18: MG, n=23), of CD4CD8DP (C, CTRL, n=18: MG, n=23) and of CD19 (D, CTRL, n=16: MG, n=22) in the spleen of grafted animals among CD45 expressing cells. E to H. FACS analysis of the expression of CD4SP (E, CTRL, n=18: MG, n=23), of CD8SP (F, CTRL, n=18: MG, n=23), of CD4CD8DP (G, CTRL, n=18: MG, n=23) and of CD19 (H, CTRL, n=16: MG, n=22) in the spleen of grafted animals among all splenocytes.

Four to six experiments are included. *p-values<0.05 and **p-values<0.01 were determined according to Student t test.

FIG. 5. MSC treatment improved MG features in the NSG-MG model. A. MSC treatment reduces MG occurrence. The Kaplan Meyer curve shows the occurrence of the disease (score>1) in the MG group (black circles, n=28), in the rMSC group (dark grey squares, n=23) and in the cMSC group (light grey triangles, n=14). B. MSC treatment reduced MG severity. Histograms represent the mean value±SEM of clinical scores attributed to each mice of each group (MG, black, n=28; rMSC, dark grey, n=23; cMSC, light grey, n=14). C. MSC treatment promoted animal weight gain. Data are normalized using each mice weight before treatment. Symbols represent the mean value±SEM of the weight change at the indicated time point for the MG group (n=18 to 20), for the rMSC group (n=16 to 19) and for the cMSC group (n=14). D. MSC treatment reduced AChR specific Abs in serum. Symbols represent the mean value±SEM of anti-AChR Abs levels before and 2 weeks after MSC treatment for the MG group (n=14), for the rMSC group (n=14) and for the cMSC group (n=10). E. MSC treatment increased muscle endplate AChR content. AChR content of the diaphragmatic muscle was measured by ¹²⁵I-αBGT labeling. Data are normalized using AChR contents of the CTRL group (levels set at 100%). Histograms correspond to the mean values±SEM in each group (MG, n=16; rMSC, n=13 and cMSC, n=11). A-E, Four to six experiments are included. B-E, *p-values<0.05 were determined according to Mann-Whitney t test. A, *p-value<0.05 were determined according to Log-rank (Mantel-Cox) test.

FIG. 6. MSC inhibited human cell proliferation in the thymus and in the spleen. Proliferating status of human cells in the xenogenic thymus (A) and in the spleen (B) was assessed by the expression of mki67 and analyzed at mRNA level by q-PCR and at protein level by IHC (C). IHC was performed on spleen section (magnification ×200; upper panel: mosaic with almost all the slide, lower panel: one representative picture) showing human cells (laminA/C positive cells) and proliferating cells (KI-67 positive cells) among all splenocytes (DAPI positive cells) in MG group (C1) and cMSC group (C2). D. mki67 mRNA expression correlated with KI-67 fluorescence intensity. Each symbol represents one mouse.

A. Four to six experiments are included (MG, n=19; rMSC, n=15; cMSC, n=13) and p-values were determined according to t-test. B. Four to six experiments are included (MG, n=19; rMSC, n=15; cMSC, n=13) and p-values were determined according to t-test. C, two experiments are included: MG, n=4; rMSC, n=3; cMSC, n=4. D, p-values were determined according to linear regression test.

FIG. 7. MSC inhibited TNF family ligand transcripts in the thymus. The TNF-α (A), BAFF (B), CD40L (C), CD40 (D), PD-L1 (E) and CD55 (F) mRNA expression was analyzed in the xenogenic thymus by q-PCR. Four to six experiments are included (MG, n=19; rMSC, n=15; cMSC, n=13) and p-values were determined according to t-test.

### DETAILED DESCRIPTION OF THE INVENTION

The present invention relates to the development of an animal model of MG. This animal model is a humanized model, said animal being grafted with human thymic tissue fragment. Advantageously, the thymic tissue is grafted under the skin of said animal. Thanks to this new procedure, bigger tissue fragments may be grafted in the animal than the procedure of the prior art involving grafting the human thymic tissue under the kidney capsule of SCID mice. Other advantages of this new humanized animal model include the ability to graft several fragments in the same animal, the easier access to and extraction of said fragments during studies, such as kinetic studies, and the overall simplification of the study of the evolution of the fragments since they are more accessible in the model of the present invention than in the model of the prior art.

The animal is a non-human animal. According to a preferred embodiment, the animal is a rodent, in particular a rat or a mouse, most preferably a mouse. In particular, the animal is an immunodeficient animal, such as an immunodeficient rodent, for example an immunodeficient mouse. Among representative immunodeficient mice known in the art, one can cite NOD, NOD/SCID, RAG, RAG2, RAG-γC and the NSG (NOD-scid IL-2Rgamma^{null}) mice. According to a preferred embodiment, the mouse is a NSG mouse, which is to date the most permissive mice to xenogeneic engraftment.

The animal may be a young animal or an adult. In a particular embodiment, the animal is a mouse of 8 to 23 weeks of age.

For generating the humanized animal model of the invention, the thymic tissue fragment is transplanted under the skin of the animal. Said fragment may be transplanted in any location part of the animal, for example in the lower back, upper back or on one or more flanks of the animal. In a particular embodiment, the fragment is transplanted in the lower back of the animal. Moreover, transplantation may be done at a single location, or at different locations.

The transplanted thymic tissue fragment volume may be comprised between 20 and 500 mm³, such as between 60 and 300 mm³. In a particular embodiment, the fragment is of around 125 mm³ (i.e. of 120, 121, 122, 123, 124, 125, 126, 127, 128, 129 or 130 mm³, most particularly 125 mm³). In a further embodiment, one or several fragments are transplanted. In particular, 1 to 5 fragments are transplanted, such as 2 to 4 fragments. Most particularly, 3 fragments are transplanted subcutaneously, for example 3 fragments transplanted in the lower back of a NSG mice, and most particularly 3 fragments of around 125 mm³ each.

The thymic tissue fragment may be one from a patient at any stage of the disease, such as early or late MG. In particular, the thymus may be from an AChR-seronegative patient, from an AChR-seropositive patient displaying low titers or from an AChR-seropositive patient displaying high titers. The patient may be any subject having MG, with no limitation with respect to the patient's age, sex or disease severity. Furthermore, the thymic tissue fragment may originate from a hyperplastic thymus, or even from a thymic tumor such as from a thymoma. In addition, the thymic tissue may be from a patient who has received a treatment for MG, such as an acetylcholinesterase inhibitor, a corticosteroid or an immunosuppressive treatment. Alternatively, the thymic tissue is from a patient who is not, or has not been, a recipient for a treatment. Preferably, the thymic tissue fragment is selected as having the lower fat ratio as possible so that grafting occurs optimally.

To produce the humanized animal model of the invention, the thymic tissue fragment is transplanted subcutaneously. Any means for transplanting tissues under the skin of an animal may be implemented in the context of the present invention. In particular, for transplantation of tissue fragment(s), one can use surgical procedure after anesthesia of the animal, optimally under a laminar flow hood, according to methods well-known in the art.

After transplantation, the animal is bred for a time sufficient for the graft to settle, before further use of the animal model. Accordingly, the animal may be bred for at least 3, 4, 5, 6, 7, 8, 9, 10, 11, 12, 13, 14, 15, 16, 17, 18, 19, 20 or 21 days, for example. Illustrative breeding times also include 1 week, 2 weeks or 3 weeks or more of breeding after MG thymus transplantation. The humanized animal model is then used for further studies by implementing the methods described below.

Grafting of the fragment may be evaluated by assessing a MG-like clinical score, as provided in the experimental part below. In particular MG-like clinical score may be assessed by observing mouse behavior and graded on a scale of 0 to 4 as follows: score 0: no sign; score 1: abnormal movements (walking with head and tail down); score 2: reduced motility; score 3: hunched posture; score 4: paralysis, dehydration or death. Animals are considered sick when they reach score 1, i.e. when they display altered movements. In addition, grafting of the MG thymic tissue may be evaluated by detecting the presence of human AChR-specific antibodies in the serum of the humanized animal model after an appropriate period as mentioned above.

According to a second aspect, the present invention relates to a method for determining the efficiency of a substance for the treatment of MG, comprising:
administering said substance to the humanized animal model of the invention; and
determining the effect of said substance in said animal model.

According to the present invention, the substance may correspond to any kind of substance potentially having a curative or preventive effect on MG. For example, the substance may be a small molecule, or a prodrug or metabolite thereof, a gene therapy product or a cell therapy product, which may be assessed thanks to the method of the present invention. A substance known for the treatment of MG, or currently used in trials for the treatment of MG, may be administered to the animal model of the invention, being thereby useful for determining whether a specific patient will potentially be responder to said therapeutic strategy.

Alternatively, the substance tested (such as a small molecule, a prodrug or metabolite thereof, a gene therapy product or cell therapy) has never been tested for the treatment of MG, and the method of the invention is therefore used as a method for screening (such as high throughput screening) substances with potential therapeutic effect on MG.

Administration of the test substance may be done via any route, such as via the oral, rectal, intramuscular, intravenous, intraarterial, intraperitoneal, cutaneous, subcutaneous or intranasal route. In addition, several substances may be tested in combination, administered either simultaneously or separately in time, in order to determine the effect of said combination, be it a synergy, an antagonism or a redundant effect.

In a particular embodiment, the substance is administered to the animal after having bred said animal after transplantation of the thymic tissue fragment according to the above disclosure. Of course, one skilled in the art will adapt the regimen to the substance that is administered, the condition of the animal model, and the route of administration. For example, the test substance may be administered a single time, the first day of the treatment, with no other administration thereof during the course of the experiment. Alternatively, the test substance may be administered several times along the method period. For example, the substance may be administered daily for the entirety of the period, once or several times a day.

Treatment efficiency may be assessed after a time sufficient for being able to observe a therapeutic effect. This time period will depend on the type of substance tested, the condition of the animal and other factors the evaluation of which is well within the knowledge of a person skilled in the art. In an illustrative embodiment, treatment efficiency is determined 1, 2, 3, 4, 5 or 6 days after administration of the test substance, or after 1, 2, 3, 4, 5, 6, 7 weeks or at least 8 weeks after administration of the test substance.

A treatment may be considered efficient when the score defined above decreases and/or when AChR-specific antibody level decreases. Alternatively, a treatment may be considered efficient when said score or said AChR-specific antibody level is stabilized by effect of the treatment, while a score calculated from a control animal (e.g. an animal model of the invention having been transplanted with a thymic tissue fragment from the same patient, and having been administered with no substance at all, or with only a composition comprising a vehicle devoid of the test substance) increases during the same time. According to another embodiment of the invention, the efficiency of the test substance is compared to the efficiency of another substance (i.e. a reference substance) known for its therapeutic effect. For example, the efficiency of the test substance may be compared to the efficiency of a corticoid (such as prednisone or hydrocortisone), of an IVIg formulation, or of a cholinesterase inhibitor (such as pyridostigminen ambenomium or neostigmine). Thanks to this embodiment, the method of the invention may be used for selecting those test substances that are more efficient than the reference substance, or at least as efficient. Alternatively, this embodiment may also allow selecting test substances that induce less secondary effects than the reference substance, a selection being possible in this case even if the test substance is less efficient in potentially treating MG than the reference substance.

In another aspect, the invention also relates to a method for evaluating functional features of the thymic tissue of a MG patient, comprising determining said functional features on the humanized animal model of the present invention. For example, features of the grafted thymic tissue fragment may be analyzed to determine the effect of a treatment against MG. Such analysis of the features of the grafted thymic tissue fragment may include a histological analysis of said fragment. According to another embodiment, a molecular analysis is carried out, wherein the presence or absence, or the level, of one or more molecules secreted by the grafted thymic tissue fragment is evaluated in the humanized animal model. The evaluation may be implemented in the thymic tissue fragment, in its vicinity, but also in other organs of the humanized animal such as in its blood, kidney, liver, spleen, muscles, central nervous system, etc. Such evaluated molecules include co-stimulatory molecules, inhibitory molecules, cytokines, chemokines, transcription factors, molecules identifying immune cells subsets, molecules linked to proliferation, for example, KI-67; TNF family ligands such as TNF-α, BAFF and/or CD40L; CD40; and CD55. The present invention provides detection of both proteins and nucleic acids, such as RNA and DNA, by any method known in the art such as by histological analysis, ELISA, western-blotting, PCR, RT-PCR, and the like. Thanks to this embodiment, molecular aspects of the graft such as protein/gene expression and other useful information may be determined.

As indicated above, the humanized animal model of the invention is useful for identifying new treatments for MG since it advantageously mimics human MG features. Accordingly, another aspect of the invention is a substance for the treatment of MG, which is identified thanks to the above described method. Strikingly, this aspect of the invention was validated by the identification of a new treatment strategy involving the administration of conditioned cells that are described below. Indeed, it is shown in the experimental part of this application that the humanized animal model of the invention has successfully allowed the identification of conditioned mesenchymal stem cells (or cMSCs) as a credible and potent therapy for MG.

Accordingly, in another aspect, the present invention relates to a conditioned mesenchymal stem cell (or cMSC) for use in a method for the treatment of an autoimmune disease, such as MG. The inventors herein surprisingly show that such conditioned mesenchymal stem cells reduce MG features in the humanized animal model of the invention, which mimics the features of the MG human disease, as compared to the effect of non-conditioned mesenchymal stem cells, which was very limited.

MSCs useful for the practice of the invention may be derived from various human tissues, including but not limited to bone marrow, cord blood, placenta and adipose tissue. In a particular embodiment, said MSCs are isolated from the bone marrow or adipose tissue of a subject, in particular from the adipose tissue.

A method of isolating mesenchymal stem cells from G-CSF mobilized peripheral blood is described by Kassis et al (Kassis, Zangi et al. 2006). A method of isolating mesenchymal stem cells from placental tissue is described by Brooke G et al. (Brooke, Rossetti et al. 2009). Methods of isolating and culturing adipose tissue, placental and cord blood mesenchymal stem cells are described by Kern et al (Kern, Eichler et al. 2006).

According to a preferred embodiment of this aspect of the present invention, the mesenchymal stem cells are human mesenchymal stem cells.

According to a particular embodiment of the invention, the cells are generated from MSCs which are autologous to the subject to be treated, i.e. the MSCs are derived from the patient to be treated, having an autoimmune disease, more particularly a MG patient.

According to another particular embodiment, the conditioned cells of the invention are ex vivo generated from MSCs which are allogenic to the subject. Representative allogenic cells will preferably include cells derived from a healthy subject, or a pool of healthy subjects. Other representative allogenic cells include commercially available MSCs, such as those marketed by Mesoblast (Prochymal MSCs).

Conditioned mesenchymal stem cells useful for the practice of the present invention may be generated by ex vivo coculturing resting MSCs (otherwise termed rMSCs in the present disclosure) (see for example (Hof-Nahor, Leshansky et al. 2012)) with peripheral blood mononuclear cells (PBMCs, such as PBMCs obtained from venous blood of healthy donors) or with monocytes, in particular with PBMCs.

The term “mesenchymal stem cell” or “MSC” is used interchangeably for adult cells which are not terminally differentiated, which can divide to yield cells that are either stem cells, or which, irreversibly differentiate to give rise to cells of a mesenchymal (chrondocyte, osteocyte and adipocyte) cell lineage.

In a particular embodiment of the invention, the MSCs and PBMCs (or monocytes) are cocultured for a time sufficient for conditioning the rMSCs. In a particular embodiment, coculture is carried out for at least 1 day, at least 2 days or at least 3 days. In particular, the coculture may be maintained during 1 to 10 days, in particular from 2 to 5 days, such as during 2, 3, 4 or 5 days. In a further particular embodiment, coculture is not done for more than 5 days. In a particular embodiment, coculture is implemented during 3 days. In a further particular embodiment, PBMCs (or monocytes) are added to the culture after rMSCs have reached an appropriate confluence, such as at least about 75% confluence, at least 80%, at least 85%, or at least 90% confluence. Preferably, coculture is done with means appropriate for preventing contact between rMSCs and PBMCs (or monocytes), but allowing diffusion of soluble mediators. Such means include culture using a cell culture insert such as a membrane, for example a transwell membrane, as provided in the experimental part of the present application. Advantageously, this embodiment allows preventing a contamination of the cMSC preparation with unwanted PBMCs (or monocytes). In another particular embodiment, the rMSCs are conditioned according to a method wherein:
a) PBMCs (or monocytes) are cultured in a cell culture medium for at least one day, such as at least two days, such as at least three days;
b) the cell culture medium is collected; and
c) said collected culture medium, devoid of PBMCs (or monocytes), is used for culturing rMSCs during the time periods provided above.

Thanks to this embodiment, soluble mediators secreted by PBMCs (or monocytes) during their culture, and therefore present in the collected medium, are used for conditioning the rMSCs. According to a specific variant of this embodiment, step a) is implemented with or without molecules of activation. According to another specific variant of this embodiment, step b) of collecting the cell medium may be done one or several times, with addition of fresh cell culture medium between each medium collection.

The cMSCs according to the invention may be used for the treatment of MG. cMSCs are administered to the patient in need thereof via any appropriate route, such as via the intramuscular, intravenous, intra-arterial or intraperitoneal route.

The cMSCs of the invention can be administered either per se or, preferably as part of a pharmaceutical composition that further comprises a pharmaceutically acceptable carrier.

As used herein a “pharmaceutical composition” refers to a preparation of one or more of the chemical conjugates described herein, with other chemical components such as pharmaceutically suitable carriers and excipients. The purpose of a pharmaceutical composition is to facilitate administration of a compound to a subject.

Hereinafter, the term “pharmaceutically acceptable carrier” refers to a carrier or a diluent that does not cause significant irritation to a subject and does not abrogate the biological activity and properties of the administered cells. Examples, without limitations, of carriers are propylene glycol; saline; emulsions; buffers; culture medium such as DMEM or RPMI; hypothermic storage medium containing components that scavenge free radicals, provide pH buffering, osmotic support, energy substrates and ionic concentrations that balance the intracellular state at low temperatures; and mixtures of organic solvents with water.

Herein the term “excipient” refers to an inert substance added to a pharmaceutical composition to further facilitate administration of a compound and maintain cell viability at a pre-determined temperature for a suitable period of time before transplantation/injection. Examples, without limitation, of excipients include albumin, plasma, serum and cerebrospinal fluid (CSF), antioxidants such as N-Acetylcysteine (NAC) or resveratrol.

According to a preferred embodiment of the present invention, the pharmaceutical carrier is an aqueous solution of buffer or a culture medium such as DMEM.

Techniques for formulation and administration of drugs may be found in “Remington's Pharmaceutical Sciences,” Mack Publishing Co., Easton, Pa., latest edition.

For any preparation used herein, the therapeutically effective amount or dose can be estimated initially from in vitro and cell culture assays. Preferably, a dose is formulated in an animal model such as the humanized animal model of the present invention, to achieve a desired concentration or titer. Such information can be used to more accurately determine useful doses in humans.

Exemplary doses of cMSCs administered to the human subject in need thereof may include 0.2×10⁶ to 5×10⁶ cells/kg, more particularly 1×10⁶ to 2×10⁶ cells/kg.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. Of course, further information may be obtained from clinical studies.

The dosage may vary depending upon the dosage form employed and the route of administration utilized. The exact formulation, route of administration and dosage can be chosen by the individual physician in view of the patient's condition, (see e.g., Fingl, et al., 1975, in “The Pharmacological Basis of Therapeutics”, Ch. 1 p. 1).

For injection, the active ingredients of the pharmaceutical composition may be formulated in aqueous solutions, preferably in physiologically compatible buffers such as Hank's solution, Ringer's solution, or physiological salt buffer and additional agents as described herein above.

Dosage amount and interval may be adjusted individually to levels of the cMSCs which are sufficient to effectively treat the disease by the administered cells. Dosages necessary to achieve the desired effect will depend on individual characteristics and route of administration.

Depending on the severity and responsiveness of the condition to be treated, dosing of cells can be of a single or a plurality of administrations, with course of treatment lasting from several days to several weeks or months depending when diminution of the disease state is achieved.

The amount of a composition to be administered will, of course, be dependent on the individual being treated, the severity of the affliction, the manner of administration, the judgment of the prescribing physician, etc. The dosage and timing of administration will be responsive to a careful and continuous monitoring of the individual changing condition.

The cells of the present invention, in at least some embodiments, may be prepackaged in unit dosage forms in a syringe ready for use. The syringe may be labeled with the name of the cells and their source. The labeling may also comprise information related to the function of the cMSCs. The syringe may be packaged in a packaging which is also labeled with information regarding the cells.

The cMSCs of the present invention, in at least some embodiments, may be coadministered with therapeutic agents useful in treating MG, such as a corticoid (such as prednisone or hydrocortisone), an IVIg formulation, a cholinessterase inhibitor (such as pyridostigminen ambenomium and neostigmine); or an immunosuppressive treatment.

In addition, numerous studies have demonstrated the strong and broad immunosuppressive properties of MSC arising them currently as a promising tool to treat autoimmune diseases. Therefore, it is expected that the properties of the MSCs conditioned according to the present invention will advantageously be implemented for treating other autoimmune diseases such as, diabetes mellitus, autoimmune thyroid diseases, multiple sclerosis, systemic lupus erythematous, rheumatoid arthritis or Sjogren's syndrome, as well as inflammatory diseases of the gut and liver such as celiac disease, Crohn's disease, and primary biliary cirrhosis.

### Examples

### Material and Methods

### Patients

All MG thymuses used for transplantation displayed follicular hyperplasia without evidence of thymoma. Clinical details of MG patients are summarized in Table 1. Control (CTRL) thymuses, obtained from newborn patients undergoing corrective heart surgery, showed no abnormality. Thymectomy was performed at the Centre Chirurgical Marie Lannelongue (Le Plessis Robinson, France) or at the Hopital Civil de Strasbourg (Strasbourg, France). All thymuses removed from patients were immediately kept in RPMI medium at 4° C. and were processed within 24 h after thymectomy.

TABLE 1

| General informations | | | | | Thymus | | | | | |
|---|---|---|---|---|---|---|---|---|---|---|
| | | | | AChR | features | Treatments | | | | |
| age | sex | score | grade | titers | #CG | corticoides | IS | anti-AChₐₛₑ | IVIg | plasmapheresis |
| 15 | F | 54 | na | >100 | some | no | no | mestinon | no | no |
| 36 | F | 85 | IIa | >100 | few | no | no | mytelase | no | no |
| 13 | F | 55 | IIIa | >100 | none | cortancyl | no | mestinon | yes | yes |
| 28 | M | 98 | IVb | >100 | few | yes | no | mestinon | no | no |
| 18 | F | 66 | IIb | >100 | some | no | no | mestinon | no | no |
| 32 | F | 68 | Iib | >100 | a lot | no | no | mestinon | yes | no |
| 24 | F | 90 | Ib | 99.3 | some | no | no | mestinon | no | no |
| 20 | M | 78 | IIa | 87.6 | few | no | no | mestinon | no | no |
| 18 | F | 65 | IIa | 57.5 | few | no | no | mestinon | yes | no |
| 15 | F | 27 | Ia | 15 | none | cortancyl | no | mestinon | yes | no |
| 19 | F | 90 | Ib | 4.23 | some | hydrocortisone | yes | mestinon | no | no |
| 27 | F | 75 | IIa | 1.14 | few | no | no | no | no | no |
| 16 | M | 74 | IIa | 0.92 | none | no | no | mestinon | yes | no |
| 22 | M | 85 | na | negative | none | no | no | mestinon | yes | no |
| 29 | F | na | na | negative | none | no | no | mestinon | no | no |
| 20 | F | na | na | negative | none | no | no | mestinon | no | no |
| 12 | M | 95 | Ia | negative | none | yes | no | mestinon | yes | no |

### Animals

NOD-scid IL-2Rgamma^{null} (NSG) mice were obtained from Charles River Laboratories (Saint-Germain-sur-l'arbresle, France). Mice were bred in our animal facilities under specific pathogen-free conditions and used between 8 to 14 weeks of age. All protocols were validated by national ethics committee (authorization number 02622.2).

### Xenogenic Thymus Transplantation

After removal of the capsule, the human thymic tissue was cut with scissors in Hanks buffer (Invitrogen, Saint-Aubin, France) in several 125 mm³ fragments and 3 of these fragments (randomly chosen) were subcutaneously transplanted in the lower back of anesthetized (80 mg/kg body weight ketamine and 4 mg/kg body weight xylazine intraperitonealy) mice. All surgical procedures were performed under laminar flow hood and aseptic conditions.

### Clinical Scoring

MG-like clinical score was assessed by observing mouse behavior and was graded on a scale of 0 to 4 as follows: score 0: no sign; score 1: abnormal movements (walking with head and tail down); score 2: reduced motility; score 3: hunched posture; score 4: paralysis, dehydration or death. Animals were considered sick when they reached score 1 i.e. when they displayed altered movements. Mice were weighted weekly and bled 2 times a month from superficial temporal vein (mandibular). Serum was collected and stored frozen. Six to eight weeks after transplantation, animals were euthanized by cervical dislocation or CO₂ inhalation. Diaphragms, xenogenic thymuses and spleens were removed and then fixed, frozen or freshly used.

### MSC Culture, Priming, and Injection

MSC were isolated from human adipose tissues, cultured and characterized as previously described (Ben-Ami, Miller et al. 2014).

In vitro MSC priming consists in a 3 days coculture with allogenic peripheral blood mononuclear cells (PBMC). Briefly, MSC were seeded into 6-well plates in DMEM/F12(HAM)1:1 (Biological industries, Beit Haemek, Israel) supplemented with FCS 10% (Eurobio, Les Ulis, France), penicillin/streptomycin 1% (Gibco, Saint-Aubin, France), and L-glutamin 1% (Gibco). Allogenic PBMC (2:1 ratio) or DMEM/F12 medium alone were added to the MSC culture when they reach about 90% confluence. PBMC were separated from adherent MSC using cell culture insert TWs (1 μm pore size, Becton Dickinson, Le Pont-de-Claix, France), to prevent cell contact but to allow diffusion of soluble mediators. PBMC were obtained from venous blood of voluntary healthy donors (Etablissement Francais du Sang, Rungis, France) using Lymphoprep density gradient centrifugation protocol (Axis-Shield, Scotland).

After 3 days of coculture, the insert containing the PBMC was removed and adherent MSC were detached using 0.25% trysin 0.01% EDTA solution for 10 minutes. 2.10⁵ to 1.10⁶ cells were then intravenously injected in mice 2 to 3 weeks after MG thymus transplantation.

### Flow Cytometry

FACS analyses were performed on cells from spleen, blood and bone marrow from grafted animals. Spleens were mechanically dissociated in PBS 3% FCS to isolate splenocytes. Bone marrow cells were collected by flushing femurs and tibiae with a PBS 3% FCS buffer using a 26-gauge needle. For spleen and blood samples, erythrocytes were removed by incubation with 1 min NH₄Cl 0.84% solution and 10 min BD Pharm Lyse (BDBioscience, Le pont de Claix, France), respectively. Single cells were then filtrated (70 μm), washed twice and stained for 30 min on ice with the following mouse monoclonal anti-human Abs combinations: CD45, LiveDead (to assay cell viability), CD4, CD8 and CD25 for T cell characterization and CD45, LiveDead, CD19, CD138 and CD20 or CD45, CD38, CD27, CD20 and IgD for B cell characterization. All antibodies are listed in Table 2. Cells were acquired on a FACSVerse (BD Bioscience) and analyzed using FlowJo software (Asland, Oreg., USA).

TABLE 2

| Abs | conjugate | host | | reactivity | clone | supplier | |
|---|---|---|---|---|---|---|---|
| CD45 | efluor450 | mouse | IgG1 | human | HI30 | eBioscience | San Diego, CA, USA |
| CD4 | FITC | mouse | IgG1 | human | MT310 | dako | Trappes, France |
| CD25 | PE | mouse | IgG1 | human | BC96 | eBioscience | San Diego, CA, USA |
| CD8 | APC | mouse | IgG2a | human | okt8 | eBioscience | San Diego, CA, USA |
| CD19 | FITC | mouse | IgG1 | human | HIB19 | eBioscience | San Diego, CA, USA |
| CD138 | PE | mouse | IgG1 | human | DL-101 | eBioscience | San Diego, CA, USA |
| CD20 | APC | mouse | IgG2b | human | 2H7 | eBioscience | San Diego, CA, USA |
| CD38 | FITC | mouse | IgG1 | human | IOB6 | immunotech | Marseille, France |
| CD27 | PE | mouse | IgG1 | human | M-T271 | BD bioscience | Le pont de Claix, |
| | | | | | | | France |
| IgD | PerCP- | mouse | IgG2a | human | IA6-2 | eBioscience | San Diego, CA, USA |
| | eFluor | | | | | | |
| | 710 | | | | | | |
| LiveDead | IR | — | — | — | — | LifeTechnologies | Saint-Aubin, France |

### Immunochemistry on Mouse Spleen and Human Thymus Sections

Cryosections (7 μm) of mouse spleens and human thymuses were collected on superfrost slides (Thermo Fisher Scientic, Braunschweig, Germany), fixed in ice-cold acetone for 20 min and blocked in a PBS 3% FCS solution to avoid unspecific binding. Sections were first stained at room temperature for 2 h with the following primary anti-human Abs: cytokeratin, fibronectin, CD21, CD4, CD8, KI-67. Ab to laminA/C was used to visualize the human cells in the mouse spleen. After 3 washes in a PBS solution, sections were next stained at room temperature for 1 h with the secondary Abs. After 3 washes in a PBS solution, sections were then stained at room temperature for 10 min with 4′,6-diamino phenylindoledihydrochloride (DAPI, LifeTechnology). All antibodies are listed in Table 3. Slides were mounted in Faramount fluorescent mounting media (Dako). Images were acquired with a Zeiss (Manly Le Roi, France) Axio Observer Z1 Inverted microscope with a ×10 eyepiece objective and a ×20 objective, using a Zeiss AxioCam MRm camera. The acquisition software was Axiovision (Zeiss).

TABLE 3

| primary | | | | | | | |
|---|---|---|---|---|---|---|---|
| Abs | conjugate | host | | reactivity | clone | supplier | |
| cytokeratin | purified | mouse | IgG1 | human | EA1/EA3 | dako | Trappes, France |
| cytokeratin | purified | mouse | IgG1 | human | MNF116 | dako | Trappes, France |
| fibronectin | purified | rabbit | — | human | polyclonal | dako | Trappes, France |
| CD21 | FITC | mouse | IgG1 | human | BL13 | immunotech | Marseille, |
| | | | | | | | France |
| CD4 | FITC | mouse | IgG1 | human | MT310 | dako | Trappes, France |
| CD8 | FITC | mouse | IgG1 | human | DK25 | dako | Trappes, France |
| KI-67 | purified | mouse | IgG1 | human | MIB-1 | dako | Trappes, France |
| KI-67 | purified | rat | IgG1 | human | 5D7 | AbCam | Cambridge, UK |
| laminA/C | purified | mouse | IgG2b | human | 636 | Leica | Newcastle, UK |

| secondary | | | | | |
|---|---|---|---|---|---|
| Abs | conjugate | host | reactivity | supplier | |
| | alexa 488 | chicken | rat | LifeTechnologies | Saint-Aubin, France |
| | alexa 488 | donkey | rabbit | LifeTechnologies | Saint-Aubin, France |
| | alexa 488 | goat | mouse | LifeTechnologies | Saint-Aubin, France |
| | alexa 594 | donkey | rat | LifeTechnologies | Saint-Aubin, France |
| | alexa 594 | chicken | mouse | LifeTechnologies | Saint-Aubin, France |
| DAPI | blue | — | — | dako | Trappes, France |

### Detection of Human Anti-AChR Abs

AChR-specific human Abs in mouse serum were detected by radioimmunoassay (RIA) as previously described (Gur-Wahnon, Mizrachi et al. 2014). Briefly, crude extracts of human muscles complexed with ¹²⁵I-α-bungarotoxin (α-BGT) were incubated with 10 μl of mouse serum. Abs were then precipitated with anti-human IgG using 2.5 μl of normal human serum as carrier IgG.

### RNA Extraction of Mouse Spleen and Human Thymus and Real-Time PCR Analysis

Frozen mouse spleens and human thymuses were disrupted with a FastPrep apparitus (QBiogen, Illkirch, France) and total RNA was extracted in TRIzol (Life technologies, Saint Aubin, France) according to the manufacturer's instruction. One μg of RNA was reverse transcribed for 1 h at 42° C. using AMV (Roche Applied Science, Mannheim, Germany) with oligo-dT (Invitrogen, Villebon sur Yvette, France). Real-time PCR reaction was performed on Light Cycler apparatus (Roche). Primers were provided by realtimeprimers.com (Elkins Park, Pa., USA) or Eurogentech. The list of the genes studied are detailed in Table 4. Spleen and thymus samples were normalized to the mean of three housekeeping genes (glucuronidase beta, peptidylpropyl isomerase and gluceraldehyde 3-phosphate dehydrogenase).

TABLE 4

| Unigene | Gene name | |
|---|---|---|
| TNFSF13 | Tumor necrosis factor (ligand) | BAFF |
| | superfamily, member 13 | |
| TNFSF13B | Tumor necrosis factor (ligand) | APRIL |
| | superfamily, member 13b | |
| BCL6 | B-cell CLL/lymphoma 6 | BCL6 |
| PRDM1 | PR domain containing 1, with ZNF domain | BLIMP1 |
| TNFRSF6 | Tumour necrosis factor receptor | TNFRSF6 |
| | superfamily, member 6 | |
| C3 | Complement component 3 | C3 |
| C5 | Complement component 5 | C5 |
| CCR5 | Chemokine (C-C motif) receptor 5 | CCR5 |
| CCR6 | Chemokine (C-C motif) receptor 6 | CCR6 |
| CCR8 | Chemokine (C-C motif) receptor 8 | CCR8 |
| CCR9 | Chemokine (C-C motif) receptor 9 | CCR9 |
| ITGAM | Integrin, alpha M | CD11b |
| ITGAX | Integrin, alpha X | CD11c |
| SDC1 | Syndecan 1 | CD138 |
| CD14 | CD14 molecule | CD14 |
| CD19 | CD19 molecule | CD19 |
| MS4A1 | Membrane-spanning 4-domains, | CD20 |
| | subfamily A, member 1 | |
| CR2 | Complement component receptor 2 | CD21 |
| CD24 | CD24 molecule | CD24 |
| IL2RA | Interleukin 2 receptor, alpha | CD25 |
| CD27 | CD27 molecule | CD27 |
| CD28 | CD28 molecule | CD28 |
| PECAM1 | In multiple clusters | CD31 |
| CD38 | CD38 molecule | CD38 |
| CD3e | CD3e molecule, epsilon | CD3 |
| CD4 | CD4 molecule | CD4 |
| CD40 | CD40 molecule | CD40 |
| CD40LG | CD40 ligand | CD40L |
| CD44 | CD44 molecule | CD44 |
| PTPRC | Protein tyrosine phosphatase, receptor type, C | CD45 |
| CD5 | CD5 molecule | CD5 |
| CD55 | CD55 molecule | CD55 |
| NCAM1 | Neural cell adhesion molecule 1 | CD56 |
| CD69 | CD69 molecule | CD69 |
| CD80 | CD80 molecule | CD80 |
| CD86 | CD86 molecule | CD86 |
| CD8A | CD8a molecule | CD8 |
| CFH | Complement factor H | CFH |
| TNNT2 | Troponin T type 2 | TNNT2 |
| CTLA4 | Cytotoxic T-lymphocyte-associated protein 4 | CTLA4 |
| CXCL13 | Chemokine (C—X—C motif) ligand 13 | CXCL13 |
| CXCR3 | Chemokine (C—X—C motif) receptor 3 | CXCR3 |
| CXCR5 | Chemokine (C—X—C motif) receptor 5 | CXCR5 |
| FAS | Fas (TNF receptor superfamily, member 6) | FAS |
| FOXP3 | Forkhead box P3 | FOXP3 |
| GATA3 | GATA binding protein 3 | GATA3 |
| CSF2 | Colony stimulating factor 2 | CSF2 |
| ICOS | Inducible T-cell co-stimulator | ICOS |
| ICOSLG | Inducible T-cell co-stimulator ligand | ICOSL |
| IFNG | Interferon, gamma | IFNG |
| IGHD | Immunoglobulin heavy constant | IGHD |
| IGHG1 | Immunoglobulin heavy constant gamma 1 | IGHG1 |
| IGHA1 | Immunoglobulin heavy constant alpha 1 | IGHA1 |
| IGHE | Immunoglobulin heavy constant epsilon | IGHE |
| IGHG3 | Immunoglobulin heavy constant gamma 3 | IGHG3 |
| IGHM | Immunoglobulin heavy constant mu | IGHM |
| IL12A | Interleukin 12A | IL12A |
| IL17A | Interleukin 17A | IL17A |
| IL1B | Interleukin 1, beta | IL1B |
| IL2 | Interleukin 2 | IL2 |
| IL21 | Interleukin 21 | IL21 |
| IL6 | Interleukin 6 | IL6 |
| IL7R | Interleukin 7 receptor | IL7R |
| IL10 | Interleukin 10 | IL10 |
| IL17RA | Interleukin 17 receptor A | IL17RA |
| IL4 | Interleukin 4 | IL4 |
| IL6R | Interleukin 6 receptor | IL6R |
| IRF4 | Interferon regulatory factor 4 | IRF4 |
| PAX5 | Paired box 5 | PAX5 |
| PDCD1 | Programmed cell death 1 | PDCD1 |
| CD274 | CD274 molecule | PD-L1 |
| PDCD1LG2 | Programmed cell death 1 ligand 2 | PD-L2 |
| RORC | RAR-related orphan receptor C | RORC |
| STAT1 | Signal transducer and activator of | STAT1 |
| | transcription 1 | |
| STAT4 | Signal transducer and activator of | STAT4 |
| | transcription 4 | |
| STAT6 | Signal transducer and activator of | STAT6 |
| | transcription 6 | |
| STAT3 | Signal transducer and activator of | STAT3 |
| | transcription 3 | |
| TBX21 | T-box 21 | t-bet |
| TGFB1 | Transforming growth factor, beta 1 | TGFB1 |
| TNF | Tumor necrosis factor | TNF |
| XBP1 | X-box binding protein 1 | XBP1 |
| MKI67 | Marker Of Proliferation Ki-67 | MKI67 |
| CCNB1 | Cyclin B1 | CCNB1 |
| CCNE1 | Cyclin E1 | CCNE1 |
| BCL2 | B-cell CLL/lymphoma 2 | BCL2 |
| CD1D | CD1d molecule | CD1D |
| ACTB | Actin, beta | hkg1 |
| B2M | Beta-2-microglobulin | hkg2 |
| GAPD | Glyceraldehyde-3-phosphate dehydrogenase | hkg3 |
| GUSB | Glucuronidase, beta | hkg4 |
| HPRT1 | Hypoxanthine phosphoribosyltransferase 1 | hkg5 |
| PGK | Phosphoglycerate kinase 1 | hkg6 |
| PPIA | Peptidylprolyl isomerase A | hkg7 |
| RPL13A | Ribosomal protein L13a | hkg8 |
| TBP | TATA-Binding Protein | hkg9 |
| TFRC | Transferrin Receptor | hkg10 |

### Endplate AChR Quantification

AChR quantification was assessed at the diaphragmatic muscular endplate using specific α-BGT binding as previously described (Aissaoui, Klingel-Schmitt et al. 1999). Briefly, diaphragms were carefully harvested from grafted mice and fixed in a PBS 4% formaldehyde solution (Sigma-Aldrich, Saint-Louis, Mo., USA). Three to five biopsies of 2 mm diameter (skin biopsy punch, helpmedical, France) were taken along the NMJ characterized by the AChE activity, visualized with the histochemical Koelle and Friedenwald reaction (Karnovsky and Roots 1964). Each biopsy was first incubated at room temperature for half an hour in a PBS 5% FCS solution, washed 3 times in a PBS 0.5% FCS solution at 4° C. for 15 min and then labeled with 0.1 μCi of ¹²⁵I-α-BGT (i.e. 4 μCi/ml, specific activity 10-20 μCi/μg, PerkinElmer, Waltham, Mass., USA) at room temperature for 15 min. Biopsies were washed again 3 times in a large volume of PBS solution at room temperature for at least 30 min and radioactivity was measured with a LB 2111 gamma counter (Berthold Technologies, Bad Wildbad, Germany). For each experiment, count per minute (cpm) values from MG group were normalized using the cpm mean values of the CTRL group (level set at 100%)

### Statistical Analysis

Differences between independent experimental groups were analyzed using GraphPad Prism 5 software (GraphPad Inc., San Diego, Calif., USA). *p-values<0.05, **p-value<0.01 and ***p-values<0.001 were determined according to Mann-Whitney t test, Student t test, linear regression test or Log-rank (Mantel-Cox) test.

### Results

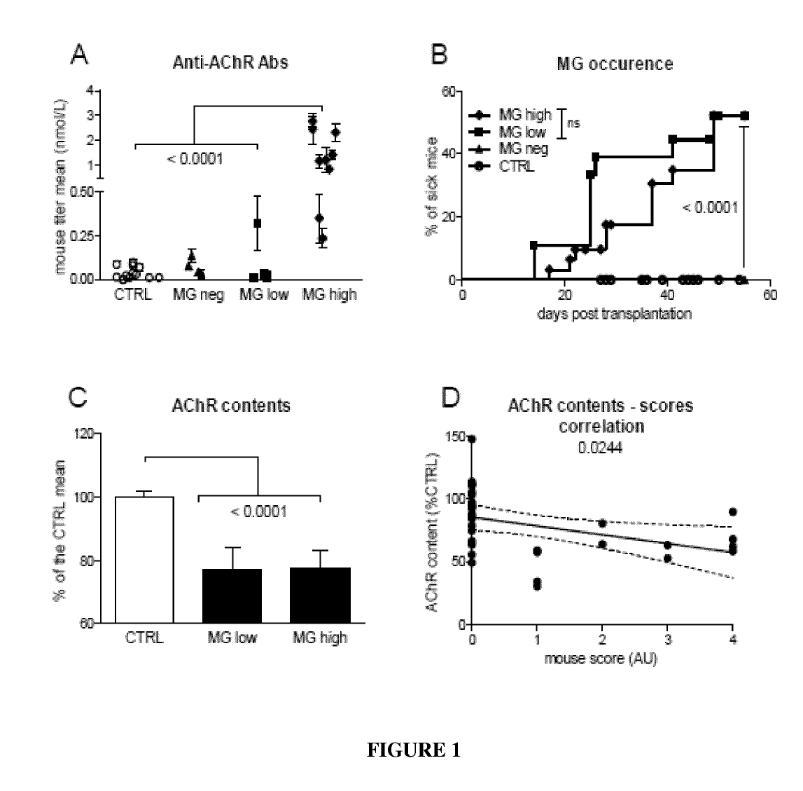

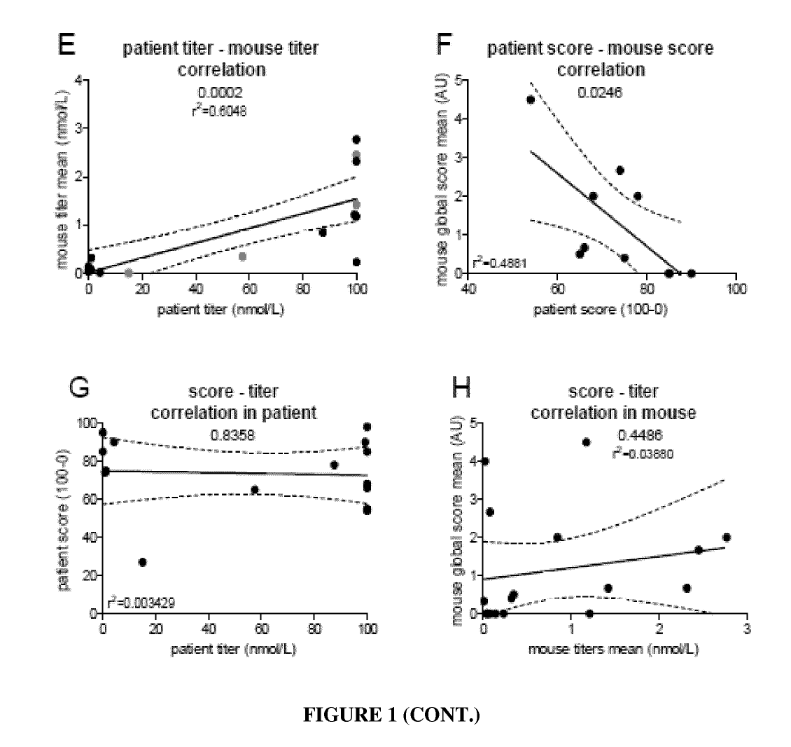

### MG Thymus Transplantation Induced MG Features (FIG. 1)

In order to develop a relevant humanized MG pre-clinical model, thymus fragments from MG patients were subcutaneously transplanted into NSG immunodeficient mice. Table 1 summarizes the clinical details of MG patients. Altogether, we performed 17 thymic grafts from MG patients and 11 from non-MG (CTRL). Four thymuses were from AChR-seronegative patients, four from AChR-seropositive patients displaying low titers and nine from AChR-seropositive patients displaying high titers. Unsurprisingly, women represented more than 70% of patients. Average age was 21.7±6.9. Sixteen patients were treated with an inhibitor of acetylcholinesterase inhibitor and four of them received also corticosteroids. One patient was treated with an acetylcholinesterase inhibitor, corticosteroids and an immunosuppressive agent. One patient had no treatment at all.

From the second week after graft, human AChR-specific Abs were detected in the serum of mice receiving thymus fragments from AChR-seropositive MG patients but not in the serum of mice receiving thymus fragments from AChR-seronegative patients or from CTRL subjects. FIG. 1A shows the mean of maximal AChR-specific Abs titers evaluated in each experimental group. Fifty percent of mice grafted with thymus fragments from both low and high titer AChR-seropositive patients displayed clinical signs (FIG. 1B) such as abnormal or reduced move and sometimes death. First symptoms occurred 2 weeks after transplantation. We did not observe any clinical sign in mice grafted with thymus fragments from AChR-seronegative patients or from non-MG CTRL. Interestingly symptom severity was fairly similar in anti-AChR high titer and low titer groups (not shown). To make the link between MG symptoms and neuromuscular junction abnormalities, we quantified AChR contents at diaphragmatic endplates. We observed an AChR loss in mice grafted with thymus fragments from both low and high titer AChR-seropositive patients in comparison to mice grafted with CTRL ones (25.8% and 26.4% reduction, respectively) (FIG. 1C). Furthermore we observed that endplate AChR loss correlated with MG clinical score (FIG. 1D). Thus, similarly to human disease, MG severity was not correlated with the anti-AChR Abs but was correlated with AChR expression loss in muscle endplates.

Additionally, we observed that mouse anti-AChR titer mean correlated with patient titer and that mouse global score mean correlated with patient score (FIGS. 1E and 1F, respectively); in other words, each mouse experiment recapitulates each patient MG features. Furthermore, in both mouse and human, AChR-specific Abs titer did not correlate with clinical score (FIGS. 1G and 1H, respectively).

Here, we demonstrate that MG thymus tissue was sufficient to induce MG symptoms in mice. As a result, our MG-NSG model truly mimicked the human disease.

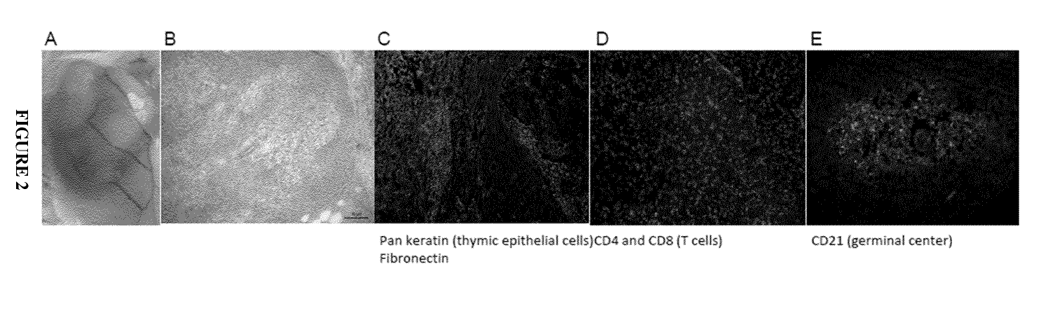

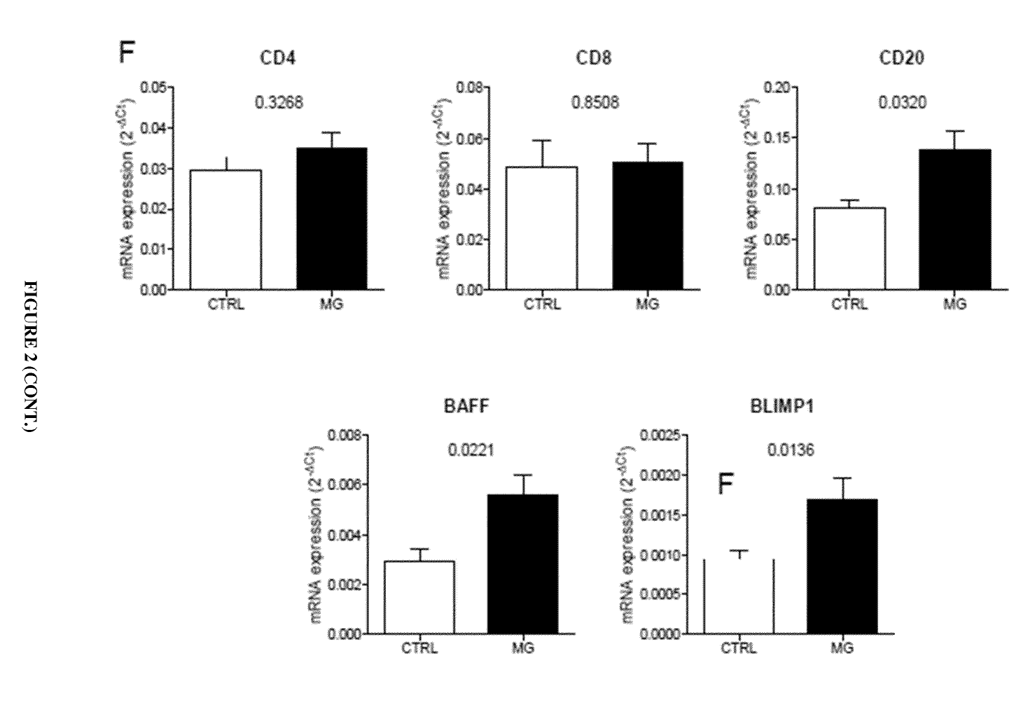

### Xenogeneic Thymus Fate (FIG. 2)

We then analyzed the fate of human thymuses in mice. We firstly noticed that new vessels had developed around thymus fragments in almost all mice of MG and CTRL groups (FIG. 2A). We also performed histological sections and observed a preserved thymic architecture, still distinguishing cortical and medullar area in lobules (FIG. 2B). IHC experiments showed many epithelial (FIG. 2C) and T cells (FIG. 2D). These data indicate that human thymus tissues were ultrastructurally preserved for at least 6 to 8 weeks after graft.

We next analyzed the transcripts of relevant genes usually involved in the physiopathology of the disease, starting with B cell related genes. Indeed, in AChR MG patients, thymus displays ectopic GC (Berrih-Aknin, Morel et al. 1987) containing large number of B cells (Berrih-Aknin, Ragheb et al. 2013). We observed in human MG thymuses a significant over expression of cd20, baff (also known as B lymphocytes stimulator, BLyS) and prdm1 (also known as blimp1) (FIG. 2F) suggesting the features of autoreactive B cell survival (Schneider, MacKay et al. 1999, Avery, Kalled et al. 2003) and maturation in Abs secreting cells (Shapiro-Shelef, Lin et al. 2003, Savitsky and Calame 2006) respectively. Furthermore, we were able to detect by IHC some GC in xenogenic thymuses of mice displaying the most obvious clinical signs (FIG. 2E), demonstrating the maintenance of the pathogenic structures.

As inflammation environment is likely to promote autoimmunity including MG (Berrih-Aknin 2014) we then analysed pro inflammatory related genes. We actually observed signs of inflammation in the xenogenic thymuses with a significant over expression of il-6, il-2, TNF-α, INF-γ and il-17 mRNA (FIG. 2G), suggesting that the thymus inflammatory environment allowed the renewal or maintenance of GC.

Thus human MG thymuses kept active pathogenic features in the NSG-MG model.

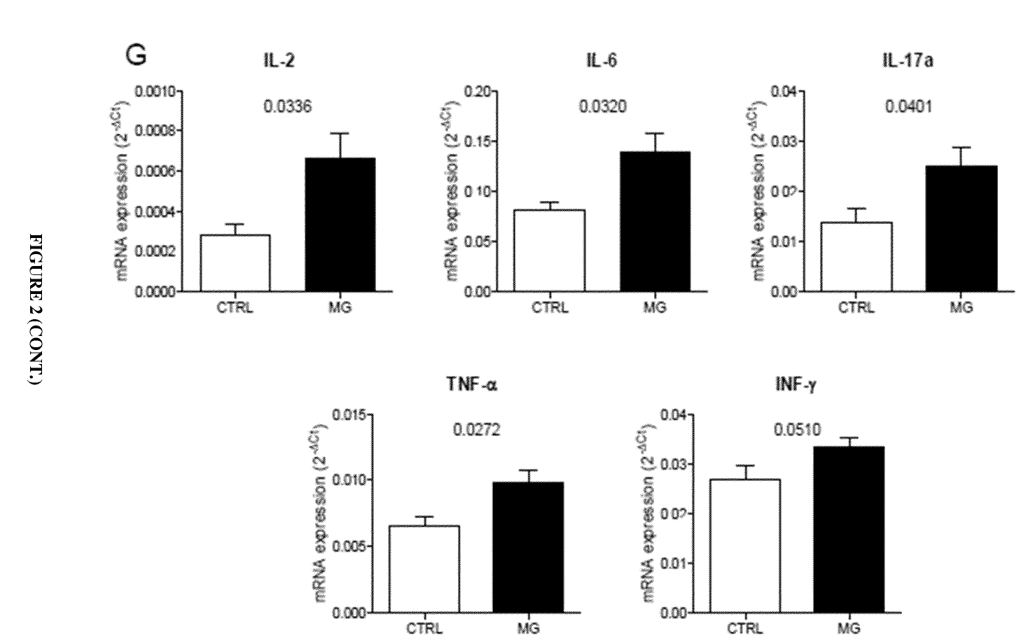

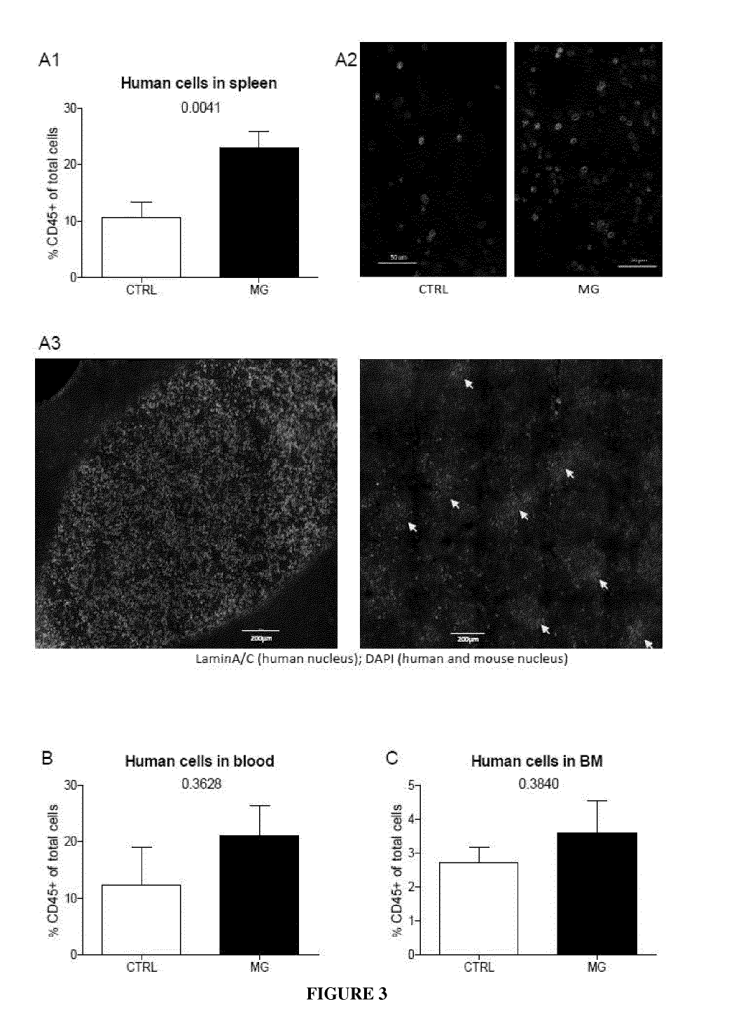

### Human Thymocytes in Periphery (FIGS. 3 & 4)

### Human Thymocytes Home to Lymphoid Organs (FIG. 3)

Since neovascularization was observed around the xenogeneic thymus, we hypothesized that human cells were able to exit from the thymus, and we investigated their ability to survive in the mouse environment. In CTRL and MG groups, human cells were detected in the spleen (evidenced with CD45 staining and FACS analysis, FIG. 3A1 and lamin A/C staining and IHC experiments, FIG. 3A2), in the blood (FIG. 3B) and in a lesser extent in the bone marrow (FIG. 3C) 6 to 8 weeks post transplantation. In the spleen they organized in kind of clusters more or less compact (FIG. 3A3). Thus human cells were able to circulate and home into mouse lymphoid organs.

Interestingly, MG group displayed more human cells than CTRL group (FIGS. 3A1, 3B and 3C), although statistical significance was reached only in the spleen (FIG. 3A1). Since MG thymus contains large number of B cells, we wondered whether MG B cells alone or all MG thymocytes could explain this high homing, and analyzed the human cell subpopulations in the mouse spleens.

### More T Cells but not More B Cells in the Spleen

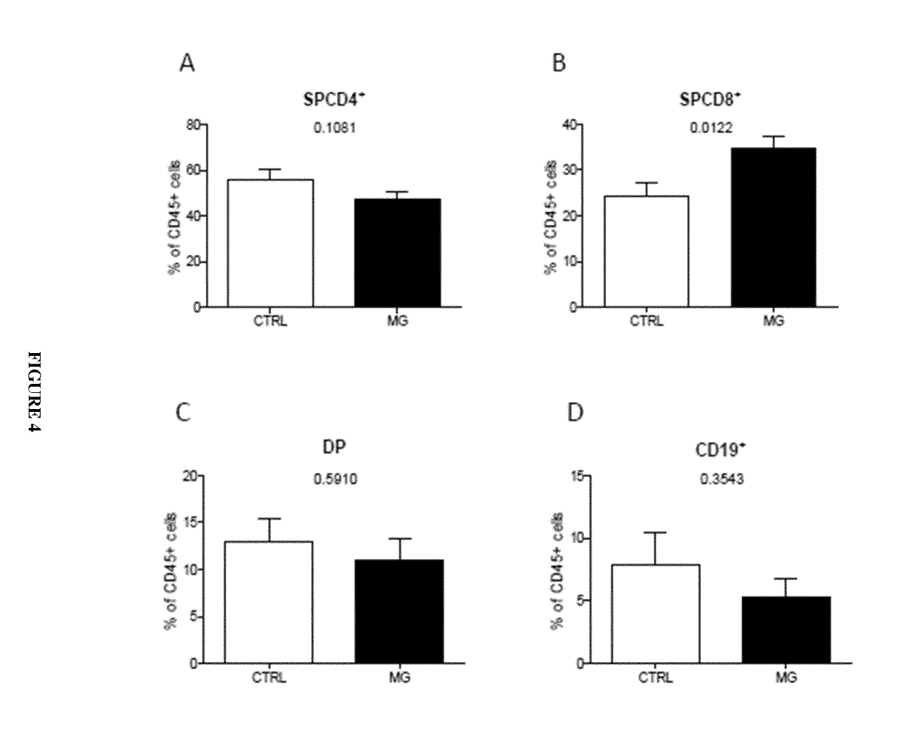

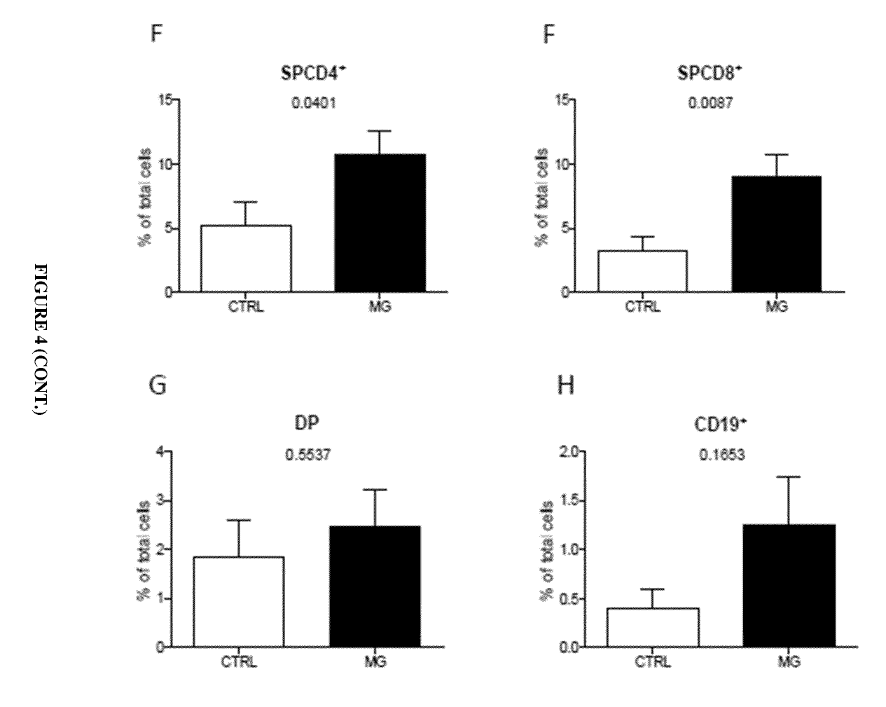

In both MG and CTRL groups, most CD45 expressing cells were CD4⁺SP cells (between 50 to 60%) and CD8⁺SP cells (20 to 30%) (FIG. 4A), meaning that mature human lymphocytes home to the periphery and suggesting that both MG and CTRL thymocytes end their differentiation in vivo.

CD4⁺SP, CD8⁺SP, CD4⁺CD8⁺DP and CD19⁺ cells in the MG group were respectively 2.06, 2.82, 1.34 and 3.09 fold more numerous in spleen compared to CTRL ones, but interestingly, only MG DP and B cells population did not significantly differ from CTRL (FIG. 2B). Since we observed an over expression of cd20 mRNA in the thymus, we could hypothesize that B cells within GC poorly migrate to the periphery.

Thus, human cells were able to keep up several weeks in grafted animals within the xenogenic thymus and/or lymphoid organs.

To summarize, we succeeded in developing a robust and humanized MG preclinical model. We then evaluated MSC as therapeutic strategy to treat MG.

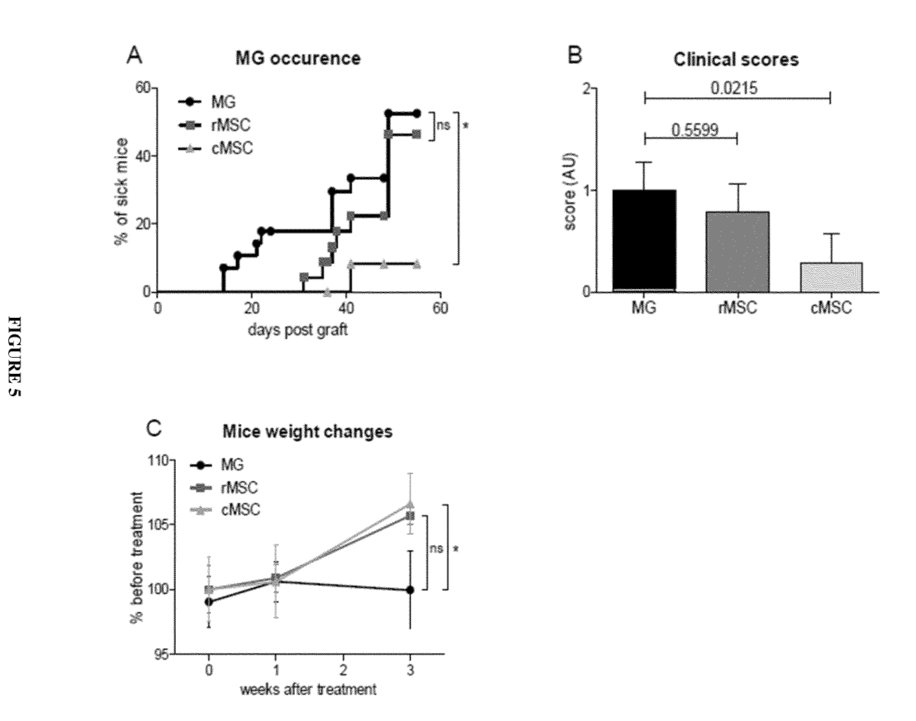

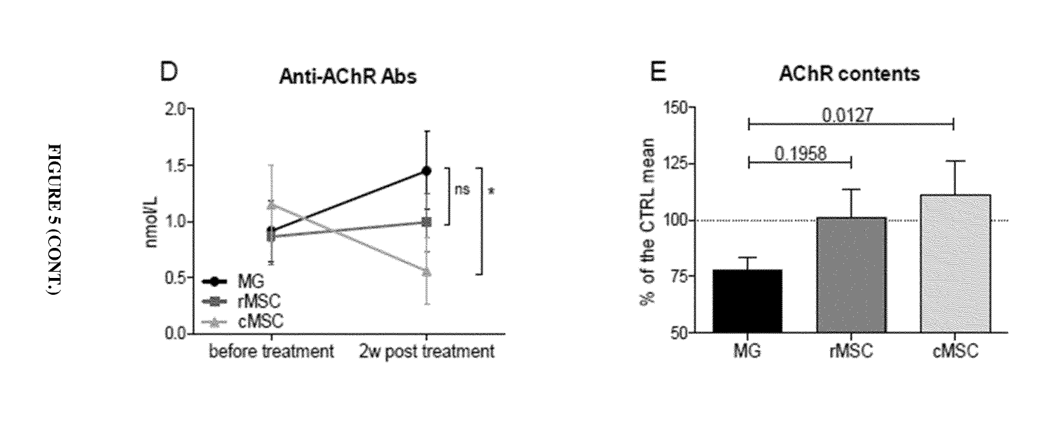

### MSC Treatment Reduced MG Features (FIG. 5)

We compared therapeutic efficacy of rMSC vs. cMSC in the MG-NSG model. Our MSC in vitro priming procedure consisted in a 3 days coculture with healthy allogenic PBMC and MSC were administrated on average at the end of the second week after graft.

In the MG group, first clinical signs occurred the second week following transplantation and 46.1% of animals were sick (score mean 1.00±0.27) the 8th week after transplantation (FIGS. 5A and 5B). Disease occurrence was slightly lower in mice injected with rMSC (39.1%) compared to untreated mice, and was delayed by about 2 weeks. MG severity in rMSC group, even slightly decreased, did not differ from MG group. However, cMSC treatment significantly decreased (4 fold) and delayed (almost 1 month) MG occurrence (12.5%) and improved clinical symptoms (score mean 0.28±0.28 compared to untreated MG mice). Mice receiving either rMSC or cMSC gained body weight during graft experiments in contrast to untreated mice (FIG. 5C). MSC effects on body weight were obvious 3 weeks after treatment. Thus MSC treatment, especially cMSC, improved MG clinical signs.

Whereas anti-AChR Abs levels increased in serum of MG group during graft experiments, rMSC and cMSC treated mice displayed stabilized and reduced titers, respectively (FIG. 5D). Furthermore, both MSC treatments had protective effect on AChR at neuromuscular junction (FIG. 5E), although cMSC were more efficient than rMSC. Altogether, MSC treatment, especially cMSC, was able to reduce anti-AChR Abs in the serum of mice, to increase the AChR expression at the diaphragmatic NMJ and, subsequently the global clinical improvement.

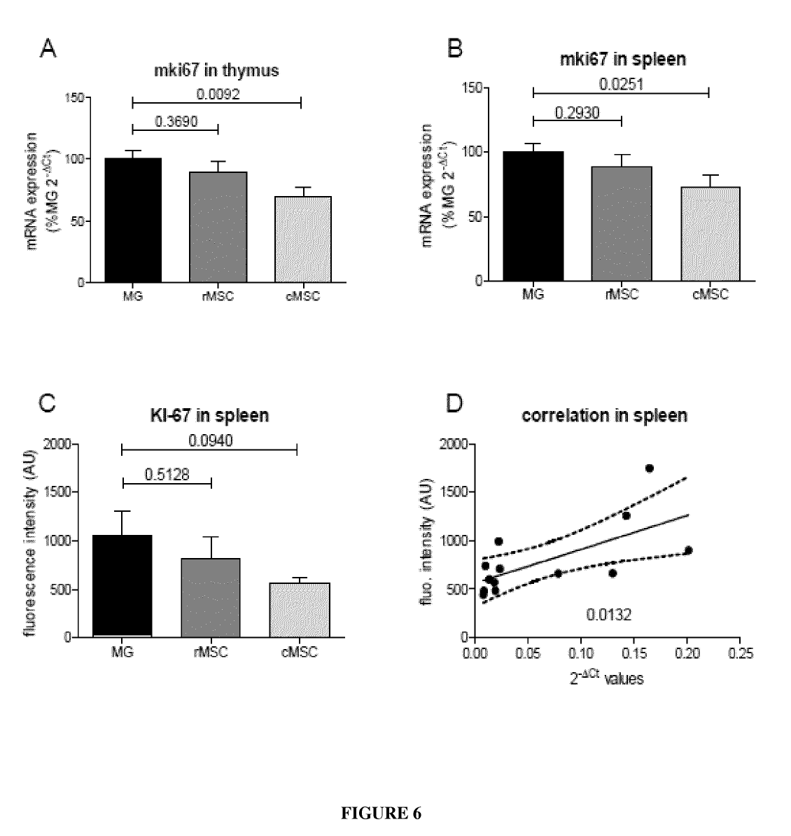

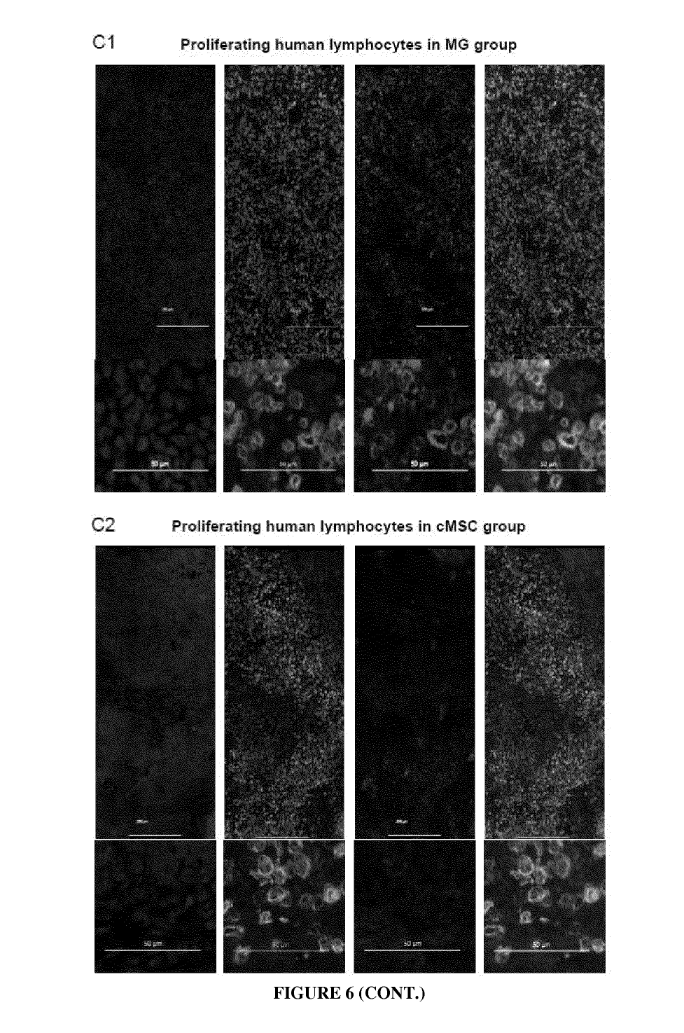

cMSC Inhibited Human Cell Proliferation in the Thymus and in the Spleen (FIG. 6)

Since MSC therapy improved the functional symptoms of MG mice, we next addressed the related mechanism.

In the thymus but also in the spleen, the transcripts of the mki67 gene that encodes a nuclear protein that is associated with cellular proliferation, was significantly diminished in cMSC group compared to MG one (FIGS. 6A and 6B). KI-67 labeling of spleen sections confirmed these data. Indeed, the intensity of fluorescence was weaker in MSC groups, especially in cMSC group, compared to MG one (FIGS. 6C, 6C1 and 6C2). Furthermore, we observed a clear correlation between the mki67 mRNA and Ki-67 protein level, analyzed in the spleen (FIG. 6D). The changes in the spleen of NSG-MG mice were more robust and reproducible when using cMSC than when using rMSCs.

These data suggest that one of the mechanisms of action of MSC in our NSG-MG model relied on the inhibition of cellular proliferation including probably pathogenic cells, and that cMSC were more efficient than rMSC.

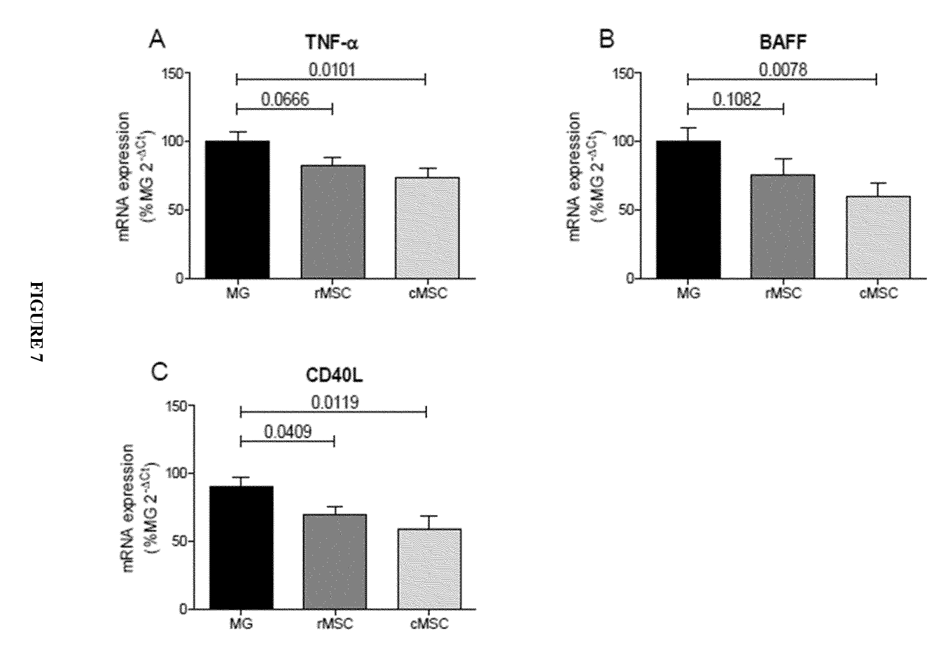

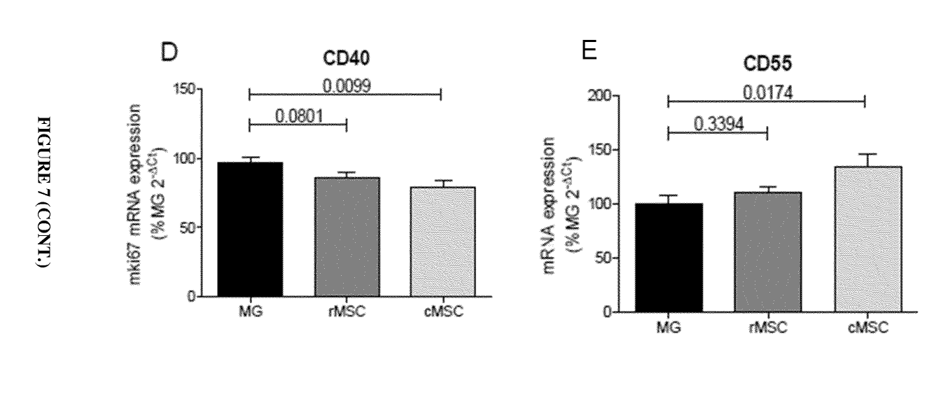

### MSC Inhibited TNF Family Ligand Transcripts in the Thymus (FIG. 7)

TNF family ligands (including TNF-α, BAFF and CD40L) play a central role in inflammation and autoimmunity (Aggarwal, Gupta et al. 2012)

We already showed an over expression of TNF-α and BAFF mRNA in MG thymuses compared to CTRL (Gradolatto, Nazzal et al. 2014) and FIG. 2. It was demonstrated that TNF-α is over expressed during EAMG development (Wang, Li et al. 2000, Duan, Wang et al. 2002) and that BAFF levels in MG patients are significantly higher compared to CTRL subjects (Kim, Yang et al. 2008, Ragheb, Lisak et al. 2008, Scuderi, Alboini et al. 2011). Furthermore, Im et al. demonstrated that CD40L blockade suppresses EAMG (Im, Barchan et al. 2001).

Here we observed that TNF-α, BAFF, CD40L and CD40 mRNA (FIG. 7 A-D) were decreased in human thymuses of both MSC treated groups compared to MG one that is interesting since two of these molecules are currently targeted in MG trials ((Rowin, Meriggioli et al. 2004, Tuzun, Meriggioli et al. 2005, Kakoulidou, Bjelak et al. 2007) and belimumab, ClinicalTrial.gov identifier: NCT01480596). So, MSC could improve MG via TNF pathway inhibition. Here also, cMSC were more efficient than rMSC to inhibit the transcription of the TNF-related molecules.

cMSC Augmented Cd55 mRNA in the Thymus (FIG. 7E)

The decay accelerating factor (DAF) regulates immune system through complement-dependent and -independent fashion (Clarke and Tenner 2014, Toomey, Cauvi et al. 2014). In the thymus and in a lesser extent in the spleen (not shown), the transcripts of the cd55 gene that encodes DAF, were augmented in the cMSC group compared to the rMSC and MG groups (FIG. 7E). As DAF deficiency was associated with autoimmunity (Toomey, Cauvi et al. 2014) including MG (Heckmann, Uwimpuhwe et al. 2010) and conversely, was shown to augment susceptibility to EAMG (Soltys, Halperin et al. 2012), cd55 mRNA augmentation in treated mice may partly explain MG improvement in our model. Here again cMSC were more efficient than rMSC to inhibit the transcription of the cd55 gene.

Altogether, our data indicate that (i) alike with clinical improvement, cMSC were more efficient than rMSC in modulating transcription of genes that are involved in MG, (ii) cellular proliferation inhibition, TNF pathway inhibition and DAF promotion could represent non-mutually exclusive mechanisms of action of MSC and (iii) MSC likely exerted their immune suppressive effects in the thymus rather than in periphery.

### Discussion

In the 90's some groups attempted to develop humanized MG models in BALB/c Scid mouse by grafting MG thymus beneath the renal capsule (Schonbeck, Padberg et al. 1992)) or adoptively infusing PBMC or PBL into the peritoneum (Vassilev, Yamamoto et al. 1999) (Martino, DuPont et al. 1993) (Wang, Karachunski et al. 1999) but without reproducing clinical signs and without being able to detect human cells in the mice.

Therefore, the present application reports the development of the first humanized animal model that credibly mimics MG features. This is an invaluable addition to the means available to those skilled in the art who will advantageously implement this animal model for studying functional features of the disease, but who will also be able to identify new therapeutic strategies thanks to this invention.

This last concept was proved in a striking manner, as the humanized model of the invention allowed us to identify a new treatment strategy involving administration of MSCs which were first conditioned according to a novel conditioning method.

Numerous studies have demonstrated the strong and broad immunosuppressive properties of MSC arising them currently as a promising tool to treat autoimmune disease. However, no entirely satisfying report was made yet showing that MSCs would actually treat autoimmune diseases, and in particular MG. Here we showed that MSC systemic administration led to MG improvement. Indeed, MG occurrence and severity were decreased in treated mice. Animals gained weight accordingly.

Importantly, and quite surprisingly, cMSC were more efficient than rMSC. It has been demonstrated that in vitro pretreatment with inflammatory cytokines, such as IFN-γ, TNF-α, IL-1 and IL-17, promotes the immunosuppressive capabilities of MSCs both in vitro (Marigo and Dazzi 2011) (Ren, Zhang et al. 2008) (Han, Yang et al. 2014) and in vivo (Polchert, Sobinsky et al. 2008) (Duijvestein, Wildenberg et al. 2011), but to an extent that was not sufficient for proposing a credible treatment of autoimmune diseases such as MG. It is herein shown that our conditioning settings did provide suitable signals to improve MSC efficiency or allow them to be easily activated in vivo.

Therefore, altogether, our results show that cMSCs prepared according to the method of the present invention represent a potent therapeutic strategy for the treatment of autoimmune diseases such as MG.

### REFERENCES

Aggarwal, B. B., S. C. Gupta and J. H. Kim (2012). “Historical perspectives on tumor necrosis factor and its superfamily: 25 years later, a golden journey.” Blood 119(3): 651-665. Aissaoui, A., I. Klingel-Schmitt, J. Couderc, D. Chateau, F. Romagne, F. Jambou, A. Vincent, P. Levasseur, B. Eymard, M. C. Maillot, P. Galanaud, S. Berrih-Aknin and S. Cohen-Kaminsky (1999). “Prevention of autoimmune attack by targeting specific T-cell receptors in a severe combined immunodeficiency mouse model of myasthenia gravis.” Ann Neurol 46(4): 559-567.
Avery, D. T., S. L. Kalled, J. I. Ellyard, C. Ambrose, S. A. Bixler, M. Thien, R. Brink, F. Mackay, P. D. Hodgkin and S. G. Tangye (2003). “BAFF selectively enhances the survival of plasmablasts generated from human memory B cells.” J Clin Invest 112(2): 286-297.
Ben-Ami, E., A. Miller and S. Berrih-Aknin (2014). “T cells from autoimmune patients display reduced sensitivity to immunoregulation by mesenchymal stem cells: Role of IL-2.” Autoimmunity Reviews 13(2): 187-196.
Berrih-Aknin, S. (2014). “Myasthenia Gravis: paradox versus paradigm in autoimmunity.” J Autoimmun 52: 1-28.
Berrih-Aknin, S. and R. Le Panse (2014). “Myasthenia gravis: a comprehensive review of immune dysregulation and etiological mechanisms.” J Autoimmun 52: 90-100.
Berrih-Aknin, S., E. Morel, F. Raimond, D. Safar, C. Gaud, J. P. Binet, P. Levasseur and J. F. Bach (1987). “The role of the thymus in myasthenia gravis: immunohistological and immunological studies in 115 cases.” Ann N Y Acad Sci 505: 50-70.
Berrih-Aknin, S., S. Ragheb, R. Le Panse and R. P. Lisak (2013). “Ectopic germinal centers, BAFF and anti-B-cell therapy in myasthenia gravis.” Autoimmun Rev 12(9): 885-893.
Brooke, G., T. Rossetti, R. Pelekanos, N. Ilic, P. Murray, S. Hancock, V. Antonenas, G. Huang, D. Gottlieb, K. Bradstock and K. Atkinson (2009). “Manufacturing of human placenta-derived mesenchymal stem cells for clinical trials.” Br J Haematol 144(4): 571-579.
Clarke, E. V. and A. J. Tenner (2014). “Complement modulation of T cell immune responses during homeostasis and disease.” J Leukoc Biol 96(5): 745-756.
Duan, R. S., H. B. Wang, J. S. Yang, B. Scallon, H. Link and B. G. Xiao (2002). “Anti-TNF-alpha antibodies suppress the development of experimental autoimmune myasthenia gravis.” J Autoimmun 19(4): 169-174.
Duijvestein, M., M. E. Wildenberg, M. M. Welling, S. Hennink, I Molendijk, V. L. van Zuylen, T. Bosse, A. C. Vos, E. S. de Jonge-Muller, H. Roelofs, L. van der Weerd, H. W. Verspaget, W. E. Fibbe, A. A. to Velde, G. R. van den Brink and D. W. Hommes (2011). “Pretreatment with interferon-gamma enhances the therapeutic activity of mesenchymal stromal cells in animal models of colitis.” Stem Cells 29(10): 1549-1558.
Gradolatto, A., D. Nazzal, F. Truffault, J. Bismuth, E. Fadel, M. Foti and S. Berrih-Aknin (2014). “Both Treg cells and Tconv cells are defective in the Myasthenia gravis thymus: roles of IL-17 and TNF-alpha.” J Autoimmun 52: 53-63.
Gur-Wahnon, D., T. Mizrachi, S. Wald-Altman, A. Al-Roof Higazi and T. Brenner (2014). “Tissue plasminogen activator involvement in experimental autoimmune myasthenia gravis: aggravation and therapeutic potential.” J Autoimmun 52: 36-43.
Han, X., Q. Yang, L. Lin, C. Xu, C. Zheng, X. Chen, Y. Han, M. Li, W. Cao, K. Cao, Q. Chen, G. Xu, Y. Zhang, J. Zhang, R. J. Schneider, Y. Qian, Y. Wang, G. Brewer and Y. Shi (2014). “Interleukin-17 enhances immunosuppression by mesenchymal stem cells.” Cell Death Differ 21(11): 1758-1768.
Heckmann, J. M., H. Uwimpuhwe, R. Ballo, M. Kaur, V. B. Bajic and S. Prince (2010). “A functional SNP in the regulatory region of the decay-accelerating factor gene associates with extraocular muscle pareses in myasthenia gravis.” Genes Immun 11(1): 1-10.
Hof-Nahor, I., L. Leshansky, S. Shivtiel, L. Eldor, D. Aberdam, J. Itskovitz-Eldor and S. Berrih-Aknin (2012). “Human mesenchymal stem cells shift CD8+ T cells towards a suppressive phenotype by inducing tolerogenic monocytes.” J Cell Sci 125(Pt 19): 4640-4650.
Im, S. H., D. Barchan, P. K. Maiti, S. Fuchs and M. C. Souroujon (2001). “Blockade of CD40 ligand suppresses chronic experimental myasthenia gravis by down-regulation of Th1 differentiation and up-regulation of CTLA-4.” J Immunol 166(11): 6893-6898.
Kakoulidou, M., S. Bjelak, R. Pirskanen and A. K. Lefvert (2007). “A clinical and immunological study of a myasthenia gravis patient treated with infliximab.” Acta Neurol Scand 115(4): 279-283.
Karnovsky, M. J. and L. Roots (1964). “A “Direct-Coloring” Thiocholine Method for Cholinesterases.” J Histochem Cytochem 12: 219-221.
Kassis, I., L. Zangi, R. Rivkin, L. Levdansky, S. Samuel, G. Marx and R. Gorodetsky (2006). “Isolation of mesenchymal stem cells from G-CSF-mobilized human peripheral blood using fibrin microbeads.” Bone Marrow Transplant 37(10): 967-976.
Kern, S., H. Eichler, J. Stoeve, H. Kluter and K. Bieback (2006). “Comparative analysis of mesenchymal stem cells from bone marrow, umbilical cord blood, or adipose tissue.” Stem Cells 24(5): 1294-1301.
Kim, J. Y., Y. Yang, J. S. Moon, E. Y. Lee, S. H. So, H. S. Lee, K. D. Park and Y. C. Choi (2008). “Serum BAFF expression in patients with myasthenia gravis.” J Neuroimmunol 199(1-2): 151-154.
Marigo, I. and F. Dazzi (2011). “The immunomodulatory properties of mesenchymal stem cells.” Semin Immunopathol 33(6): 593-602.
Martino, G., B. L. DuPont, R. L. Wollmann, P. Bongioanni, J. Anastasi, J. Quintans, B. G. Amason and L. M. Grimaldi (1993). “The human-severe combined immunodeficiency myasthenic mouse model: a new approach for the study of myasthenia gravis.” Ann Neurol 34(1): 48-56.
Polchert, D., J. Sobinsky, G. Douglas, M. Kidd, A. Moadsiri, E. Reina, K. Genrich, S. Mehrotra, S. Setty, B. Smith and A. Bartholomew (2008). “IFN-gamma activation of mesenchymal stem cells for treatment and prevention of graft versus host disease.” Eur J Immunol 38(6): 1745-1755.
Ragheb, S., R. Lisak, R. Lewis, G. Van Stavern, F. Gonzales and K. Simon (2008). “A potential role for B-cell activating factor in the pathogenesis of autoimmune myasthenia gravis.” Arch Neurol 65(10): 1358-1362.
Ren, G., L. Zhang, X. Zhao, G. Xu, Y. Zhang, A. I. Roberts, R. C. Zhao and Y. Shi (2008). “Mesenchymal stem cell-mediated immunosuppression occurs via concerted action of chemokines and nitric oxide.” Cell Stem Cell 2(2): 141-150.
Rowin, J., M. N. Meriggioli, E. Tuzun, S. Leurgans and P. Christadoss (2004). “Etanercept treatment in corticosteroid-dependent myasthenia gravis.” Neurology 63(12): 2390-2392.
Savitsky, D. and K. Calame (2006). “B-1 B lymphocytes require Blimp-1 for immunoglobulin secretion.” J Exp Med 203(10): 2305-2314.
Schneider, P., F. MacKay, V. Steiner, K. Hofmann, J. L. Bodmer, N. Holler, C. Ambrose, P. Lawton, S. Bixler, H. Acha-Orbea, D. Valmori, P. Romero, C. Werner-Favre, R. H. Zubler, J. L. Browning and J. Tschopp (1999). “BAFF, a novel ligand of the tumor necrosis factor family, stimulates B cell growth.” J Exp Med 189(11): 1747-1756.
Schonbeck, S., F. Padberg, R. Hohlfeld and H. Wekerle (1992). “Transplantation of thymic autoimmune microenvironment to severe combined immunodeficiency mice. A new model of myasthenia gravis.” J Clin Invest 90(1): 245-250.
Scuderi, F., P. E. Alboini, E. Bartoccioni and A. Evoli (2011). “BAFF serum levels in myasthenia gravis: effects of therapy.” J Neurol 258(12): 2284-2285.
Shapiro-Shelef, M., K. I. Lin, L. J. McHeyzer-Williams, J. Liao, M. G. McHeyzer-Williams and K. Calame (2003). “Blimp-1 is required for the formation of immunoglobulin secreting plasma cells and pre-plasma memory B cells.” Immunity 19(4): 607-620.
Soltys, J., J. A. Halperin and Q. Xuebin (2012). “DAF/CD55 and Protectin/CD59 modulate adaptive immunity and disease outcome in experimental autoimmune myasthenia gravis.” J Neuroimmunol 244(1-2): 63-69.
Toomey, C. B., D. M. Cauvi and K. M. Pollard (2014). “The role of decay accelerating factor in environmentally induced and idiopathic systemic autoimmune disease.” Autoimmune Dis 2014: 452853.
Tuzun, E., M. N. Meriggioli, J. Rowin, H. Yang and P. Christadoss (2005). “Myasthenia gravis patients with low plasma IL-6 and IFN-gamma benefit from etanercept treatment.” J Autoimmun 24(3): 261-268.
Vassilev, T., M. Yamamoto, A. Aissaoui, E. Bonnin, S. Berrih-Aknin, M. D. Kazatchkine and S. V. Kaveri (1999). “Normal human immunoglobulin suppresses experimental myasthenia gravis in SCID mice.” Eur J Immunol 29(8): 2436-2442.
Wang, H. B., H. Li, F. D. Shi, B. J. Chambers, H. Link and H. G. Ljunggren (2000). “Tumor necrosis factor receptor-1 is critically involved in the development of experimental autoimmune myasthenia gravis.” Int Immunol 12(10): 1381-1388.
Wang, Z. Y., P. I. Karachunski, J. F. Howard, Jr. and B. M. Conti-Fine (1999). “Myasthenia in SCID mice grafted with myasthenic patient lymphocytes: role of CD4+ and CD8+ cells.” Neurology 52(3): 484-497.

1-15. (canceled)

16. A method for generating conditioned mesenchymal stem cell (cMSCs) useful for the treatment of Myasthenia Gravis (MG), comprising coculturing resting MSCs (rMSCs) with peripheral blood mononuclear cells (PBMCs).

17. The method according to claim 16, wherein said rMSCs originate from bone marrow or adipose tissue.

18. The method according to claim 16, wherein said PBMCs originate from venous blood of a healthy donor.

19. The method according to claim 16, wherein coculture is carried out for at least 1 day, at least 2 days or at least 3 days.

20. A method of treating an autoimmune disease comprising administering a conditioned mesenchymal stem cell produced according to the method of claim 16 to a subject having an autoimmune disease.

21. The method according to claim 20, wherein said autoimmune disease is MG, diabetes mellitus, autoimmune thyroid diseases, multiple sclerosis, systemic lupus erythematous, rheumatoid arthritis, Sjogren's syndrome, an inflammatory disease of the gut and liver, celiac disease, Crohn's disease, or primary biliary cirrhosis.

22. A pharmaceutical composition comprising cMSCs obtained according to the method of claim 16.

23. A humanized animal model of Myasthenia Gravis (MG), wherein a fragment of a human thymic tissue from a MG patient is transplanted subcutaneously in a non-human immunodeficient animal.

24. The model according to claim 23, wherein the animal is a rodent.

25. The model according to claim 23, wherein the immunodeficient animal is a mouse.

26. The model according to claim 23, wherein the immunodeficient animal is an NOD-scid IL-2Rgamma^{null} (NSG) mouse.

27. The model according to claim 23, wherein the transplanted thymic tissue fragment has a volume between 20 and 500 mm³.

28. The model according claim 23, wherein from 1 to 5 fragments are transplanted into said animal.

29. A method for determining the efficiency of a substance for the treatment of MG, comprising:
administering said substance to the humanized animal model according to claim 23; and
determining the effect of said substance in said model.

30. The method according to claim 29, wherein the substance is administered at least 1, 2 or 3 weeks after MG thymic tissue transplantation into said model.

31. The method according to claim 29, wherein substance efficiency is determined 1, 2, 3, 4, 5 or 6 days after administration of the substance, or after 1, 2, 3, 4, 5, 6, 7 weeks or at least 8 weeks after administration of said substance.

32. A method for evaluating functional features of the thymic tissue of a MG patient, comprising determining said functional features on the humanized animal model according to claim 23.
